(19) United States
(12) Patent Application Publication
(10) Pub. No.: US 2024/0376575 A1
(43) Pub. Date: Nov. 14, 2024

(54) MAGNETIC BEAD AND MAGNETIC BEAD REAGENT

(71) Applicant: SEIKO EPSON CORPORATION, TOKYO (JP)

(72) Inventors: Fumio TAKAGI, CHINO-SHI (JP); Michio NAKAMORI, Matsumoto-shi (JP)

(21) Appl. No.: 18/655,460

(22) Filed: May 6, 2024

(30) Foreign Application Priority Data
May 8, 2023 (JP) 2023-076553

(51) Int. Cl. C22C 38/02; C22C 38/18; C23C 16/40; C23C 16/455; G01N 1/40; G01N 21/73; H01F 1/147

(52) U.S. Cl. CPC C22C 38/02; C22C 38/18; C23C 16/401; G01N 1/4044; G01N 21/73; H01F 1/14766; C22C 2200/02; C23C 16/45525

(57) ABSTRACT

A magnetic bead includes: a magnetic metal particle; and a coating layer that covers a surface of the magnetic metal particle, contains an inorganic oxide, and has an average thickness of 15 nm or more, in which, when the magnetic bead is subjected to an operation of mixing 0.1 g of ethylenediaminetetraacetic acid (EDTA), 5 mL of pure water, and 100 mg of the magnetic bead to prepare a first mixture, an operation of mixing the first mixture with 5 mL of hydrochloric acid having a concentration of 10 mM to obtain a second mixture, and then stirring the second mixture with a vortex mixer for 30 minutes, and an operation of allowing the stirred second mixture to stand for one day, then collecting a supernatant, and measuring a metal ion concentration of the supernatant by high-frequency inductively coupled plasma (ICP) emission spectroscopy, the metal ion concentration of the supernatant is 0.01 ppm or more and 5.00 ppm or less.

The present application is based on, and claims priority from JP Application Serial Number 2023-076553, filed May 8, 2023, the disclosure of which is hereby incorporated by reference herein in its entirety.

### BACKGROUND

### 1. Technical Field

The present disclosure relates to a magnetic bead and a magnetic bead reagent.

### 2. Related Art

In recent years, in the fields of diagnosis and biological science in the medical field, there is an increasing demand for test of a biological material. Among biological material test techniques, a polymerase chain reaction (PCR) method is a method of extracting a nucleic acid such as deoxyribonucleic acid (DNA) or ribonucleic acid (RNA), specifically amplifying and detecting the nucleic acid. In such a process of testing the biological material, it is first necessary to extract a substance to be tested from a specimen. For the extraction of the biological material, a magnetic separation method using a magnetic bead is widely used. In the magnetic separation method, a biological material is extracted by applying a magnetic field using a magnetic bead having a function of binding the biological material to be extracted. Specifically, a magnetic bead having a function of binding a substance to be tested on a surface thereof is dispersed in a dispersion medium, then an obtained dispersion liquid is attached to a magnetic field generation apparatus such as a magnetic stand, and ON and OFF of magnetic field application is repeated a plurality of times. Accordingly, the substance to be tested is extracted. Since such a magnetic separation method is a method of separating and collecting the magnetic bead by a magnetic force, a rapid separation operation can be performed.

The same magnetic separation method is used not only in extraction for the PCR method but also in fields of protein purification, separation and extraction of exosomes and cells, and the like.

For example, JP-A-2010-156054 discloses, as a metal microparticle for a magnetic bead, a metal microparticle in which a magnetic metal particle core containing a magnetic metal as a main component and having an average particle diameter of 10 μm or less is covered in multiple layers with two or more different inorganic materials. According to such a metal microparticle, since the magnetic metal particle core is covered with the inorganic materials in multiple layers, elution of the metal into a solvent can be prevented.

JP-A-2010-156054 is an example of the related art.

However, in the metal microparticle disclosed in JP-A-2010-156054, the magnetic metal particle core is covered with the inorganic materials in multiple layers. Therefore, the metal microparticle has a low movement speed when attracted by an applied magnetic field, and it is time-consuming for magnetic separation. Meanwhile, as an amount of the inorganic materials covering the magnetic metal particle core decreases, an elution amount of the metal may increase. As the elution amount of the metal increases, the eluted metal inhibits extraction and collection of the biological material.

Therefore, it is a problem to obtain a magnetic bead that can rapidly perform magnetic separation and has high biological material extraction efficiency.

### SUMMARY

A magnetic bead according to an application example of the disclosure is a magnetic bead containing:
a magnetic metal particle; and
a coating layer that covers a surface of the magnetic metal particle, contains an inorganic oxide, and has an average thickness of 15 nm or more, in which
when the magnetic bead is subjected to
   an operation of mixing 0.1 g of ethylenediaminetetraacetic acid (EDTA), 5 mL of pure water, and 100 mg of the magnetic bead to prepare a first mixture, an operation of mixing the first mixture with 5 mL of hydrochloric acid having a concentration of 10 mM to obtain a second mixture, and then stirring the second mixture with a vortex mixer for 30 minutes, and
   an operation of allowing the stirred second mixture to stand for one day, then collecting a supernatant, and measuring a metal ion concentration of the supernatant by high-frequency inductively coupled plasma (ICP) emission spectroscopy,
the metal ion concentration of the supernatant is 0.01 ppm or more and 5.00 ppm or less.

A magnetic bead reagent according to an application example of the disclosure includes:
the magnetic bead according to the application example of the disclosure; and
a dispersion medium in which the magnetic bead is dispersed.

### BRIEF DESCRIPTION OF THE DRAWINGS

FIG. 1 is a flowchart showing an example of a biological material extraction method.

FIG. 2 is a schematic diagram showing the biological material extraction method shown in FIG. 1.

FIG. 3 is a schematic diagram showing the biological material extraction method shown in FIG. 1.

FIG. 4 is a schematic diagram showing the biological material extraction method shown in FIG. 1.

FIG. 5 is a schematic diagram showing the biological material extraction method shown in FIG. 1.

FIG. 6 is a schematic diagram showing the biological material extraction method shown in FIG. 1.

FIG. 7 is a cross-sectional view showing a magnetic bead according to an embodiment.

FIG. 8 is a cross-sectional view showing a modification of the magnetic bead in FIG. 7.

### DESCRIPTION OF EMBODIMENTS

Hereinafter, a preferred embodiment of a magnetic bead and a magnetic bead reagent according to the disclosure will be described in detail with reference to the accompanying drawings.

The magnetic bead according to the embodiment is a particle group which adsorbs a biological material and which is used for magnetic separation. Magnetic separation is a technique of applying an external magnetic field to a container in which a solid phase containing a magnetic bead and a liquid phase containing a dispersion medium are charged to magnetically attract the solid phase and thus separating the solid phase from the liquid phase.

The biological material refers to a substance such as a nucleic acid such as DNA or RNA, a protein, a saccharide, various cells such as a cancer cell, a peptide, a bacterium, and a virus. The nucleic acid may be present in a state of being contained in a biological sample such as a cell or biological tissue, a virus, or a bacterium. Such a biological material is extracted through, for example, each step of dissolution, adsorption, washing, and elution, and is used for an test or the like. By magnetically separating the magnetic bead where the biological material is adsorbed, purification and extraction of the biological material can be easily performed.

### 1. Biological Material Extraction Method

Hereinafter, an example of a biological material extraction method using magnetic separation will be described. In the following description, a case where the biological material is a nucleic acid will be described as an example.

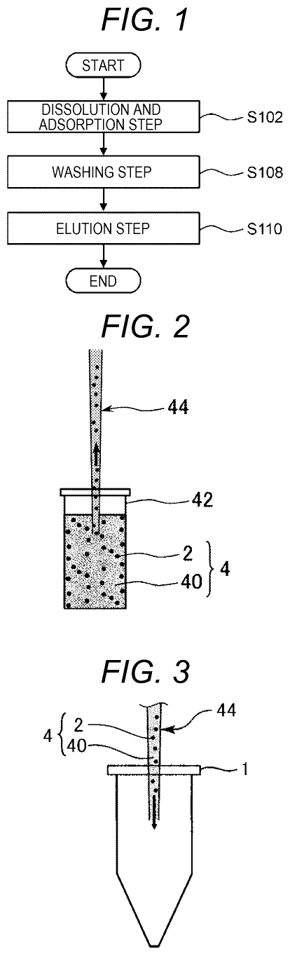

FIG. 1 is a flowchart showing an example of the biological material extraction method. FIGS. 2 to 6 are schematic diagrams showing the biological material extraction method shown in FIG. 1.

The biological material extraction method shown in FIG. 1 includes a dissolution and adsorption step S102, a washing step S108, and an elution step S110. Hereinafter, each step will be sequentially described.

### 1.1. Dissolution and Adsorption Step

In the dissolution and adsorption step S102, first, as shown in FIG. 2, a magnetic bead dispersion liquid 4 (magnetic bead reagent) containing a magnetic bead 2 and a dispersion medium 40 is prepared in a container 42.

In the container 42, the magnetic bead dispersion liquid 4 is sufficiently stirred. Accordingly, the magnetic bead 2 is uniformly dispersed in the magnetic bead dispersion liquid 4. Thereafter, a predetermined amount of the magnetic bead dispersion liquid 4 is collected from the container 42 by a pipette 44 or the like and dispensed into a container 1 shown in FIG. 3. If concentration distribution of the magnetic bead dispersion liquid 4 is uniform, a target amount of the magnetic bead 2 is added to the container 1 by the dispensing.

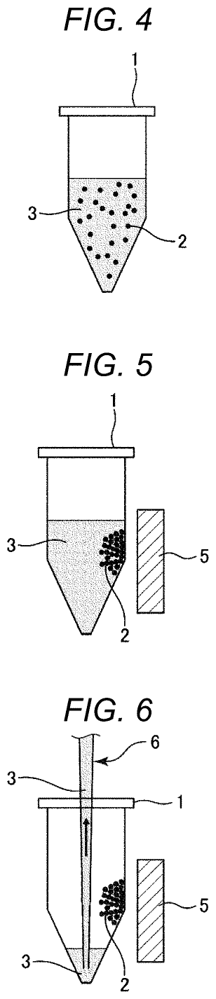

Next, a specimen sample, which contains a nucleic acid, and a lysis and bindin buffer are charged into the container 1 shown in FIG. 3. Then, contents in the container 1 are mixed. Accordingly, the magnetic bead 2 is dispersed in a liquid 3 in the container 1 as shown in FIG. 4. Since the nucleic acid is usually contained in a cell membrane and a nucleus, the nucleic acid is extracted by dissolving and removing the cell membrane and a so-called outer shell of the nucleus by a dissolution action of the lysis and bindin buffer. Thereafter, the nucleic acid is adsorbed to each magnetic bead 2 by an adsorption action of the lysis and bindin buffer.

As the lysis and bindin buffer, for example, a liquid containing a chaotropic substance is used. The chaotropic substance has a function of generating chaotropic ions in an aqueous solution, reducing an interaction between water molecules, and thereby destabilizing a structure, and thus contributes to adsorption of the nucleic acid to the magnetic bead 2.

Particularly, when RNA among nucleic acids is extracted, it is preferable to acidify the liquid 3 in the container 1 by adding an acid or the like. An RNA monomer contains ribose and thus is more soluble in a polar solvent than DNA. When RNA and DNA are separated using such a difference, the liquid 3 may be acidified by, for example, adding an acid after the outer shell is dissolved and removed. Thereafter, a non-polar solvent such as phenol or chloroform is added to the liquid 3. Accordingly, DNA migrates to the non-polar solvent whereas RNA remains in the polar solvent. As a result, RNA and DNA can be separated, and RNA can be extracted.

When RNA is extracted, a pH of the liquid 3 in the container 1 is preferably 5.0 or less, more preferably 2.0 or more and 4.0 or less. Accordingly, an ionization equilibrium of a phosphate group contained in RNA is biased to a hydroxy group, and thus RNA is particularly easily dissolved in the polar solvent. Therefore, extraction efficiency of RNA can be further improved.

### 1.1.1. Magnetic Separation Operation

In the dissolution and adsorption step S102, an external magnetic field acts on the magnetic bead 2 where the nucleic acid is adsorbed, and magnetic attraction occurs. Accordingly, the magnetic bead 2 is moved to an inner wall of the container 1 and fixed. As a result, as shown in FIG. 5, the magnetic bead 2 as a solid phase and the liquid 3 as a liquid phase can be separated. In the specification, such an operation of fixing the magnetic bead 2 by applying an external magnetic field is referred to as a “magnetic separation operation”.

Before the magnetic separation operation, the contents in the container 1 are stirred as necessary. Accordingly, a probability that the nucleic acid is adsorbed by the magnetic bead 2 increases. In the stirring, for example, a vortex mixer, hand shaking, or pipetting is used.

In the application of the external magnetic field, for example, a magnet 5 disposed beside the container is used. The magnet 5 may be an electromagnet or a permanent magnet. When the external magnetic field acts on the magnetic bead 2, the magnetic bead 2 moves toward the magnet 5.

### 1.1.2. Liquid Discharge Operation

In the dissolution and adsorption step S102, in a state in which the magnetic bead 2 is fixed to the inner wall of the container 1, as shown in FIG. 6, the liquid 3 accumulated at a bottom of the container 1 is suctioned and discharged by, for example, a pipette 6. In the specification, such an operation of discharging the liquid 3 is referred to as a “liquid discharge operation”. By the liquid discharge operation, the magnetic bead 2 where the nucleic acid is adsorbed remains in the container 1.

After the liquid discharge operation, an acceleration may be applied to the container 1 as necessary. Accordingly, the liquid 3 adhering to the magnetic bead 2 can be shaken off, and the unseparated liquid 3 can be reduced. The acceleration may be a centrifugal acceleration. A centrifugal separator may be used to apply the centrifugal acceleration.

### 1.2. Washing Step

In the washing step S108, the magnetic bead 2 where the nucleic acid is adsorbed is washed. Washing refers to an operation of removing contaminants by bringing the magnetic bead 2 where the nucleic acid is adsorbed into contact with a washing liquid and then separating the magnetic bead 2 from the washing liquid again in order to remove the contaminants adsorbed at the magnetic bead 2.

Specifically, after the washing liquid is charged into the container 1 in which the magnetic bead 2 where the nucleic acid is adsorbed is charged, the magnetic separation operation and the liquid discharge operation are performed again.

Among these, in the magnetic separation operation, first, the washing liquid is supplied into the container 1 by a pipette or the like. Then, the magnetic bead 2 and the washing liquid are stirred. Accordingly, the washing liquid comes into contact with the magnetic bead 2, and the magnetic bead 2 where the nucleic acid is adsorbed is washed. In the stirring, for example, a vortex mixer, hand shaking, or pipetting is used. At this time, the application of the external magnetic field may be temporarily turned off. Accordingly, since the magnetic bead 2 is redispersed in the washing liquid, washing efficiency can be further improved.

Next, as the liquid discharge operation, the washing liquid pooled at the bottom of the container 1 is discharged in a state in which the magnetic bead 2 is fixed to the inner wall of the container 1. The magnetic bead 2 is washed by performing the above-described supply and discharge of the washing liquid at least once. Accordingly, the contaminants can be removed with high accuracy.

The washing liquid is not particularly limited as long as the washing liquid is a liquid that does not promote elution of the nucleic acid and does not promote binding of the contaminants to the magnetic bead 2, and examples thereof include organic solvents such as ethanol, isopropyl alcohol, and acetone, aqueous solutions thereof, and low-salt-concentration aqueous solutions thereof.

The washing liquid may contain a surfactant such as Triton (registered trademark), Tween (registered trademark), or SDS. The washing liquid may contain a chaotropic substance such as guanidine hydrochloride.

The washing step S108 may be performed as necessary and may be omitted when washing is not necessary.

### 1.3. Elution Step

In the elution step S110, the nucleic acid adsorbed at the magnetic bead 2 is eluted into an elution buffer. The elution is an operation of transferring the nucleic acid to the elution buffer by bringing the magnetic bead 2 where the nucleic acid is adsorbed into contact with the elution buffer and then separating the magnetic bead 2 from the elution buffer again.

Specifically, after the elution buffer is charged into the container 1 in which the magnetic bead 2 where the nucleic acid is adsorbed is charged, the magnetic separation operation and the liquid discharge operation are performed again.

Among these, in the magnetic separation operation, first, the elution buffer is supplied into the container 1 by a pipette or the like. Then, the magnetic bead 2 and the elution buffer are stirred. Accordingly, the elution buffer comes into contact with the magnetic bead 2, and the nucleic acid is eluted into the elution buffer. In the stirring, for example, a vortex mixer, hand shaking, or pipetting is used. At this time, the application of the external magnetic field may be temporarily turned off. Accordingly, since the magnetic bead 2 is dispersed in the elution buffer, elution efficiency can be further improved.

Next, as the liquid discharge operation, the elution buffer pooled at the bottom of the container 1 is discharged in a state in which the magnetic bead 2 is fixed to the inner wall of the container 1. Accordingly, the elution buffer containing the nucleic acid can be collected.

The elution buffer is not particularly limited as long as the elution buffer is a liquid that promotes the elution of the nucleic acid from the magnetic bead 2 where the nucleic acid is adsorbed, and for example, in addition to water such as sterilized water or pure water, a TE buffer, that is, an aqueous solution which contains 10 mM of Tris-HCl buffer and 1 mM of EDTA and which has a pH of about 8 is preferably used.

The elution buffer may contain a surfactant such as Triton (registered trademark), Tween (registered trademark), or SDS. In addition, sodium azide may be contained as a preservative.

In the elution step S110, the elution buffer may be heated. Accordingly, the elution of the nucleic acid can be promoted. A heating temperature of the elution buffer is not particularly limited, and is preferably 70° C. or higher and 200° C. or lower, more preferably 80° C. or higher and 150° C. or lower, and still more preferably 95° C. or higher and 125° C. or lower.

### 2. Magnetic Bead

Next, the magnetic bead 2 will be described. The magnetic bead 2 is a particle that has magnetism and whose surface exhibits a binding affinity with a biological material.

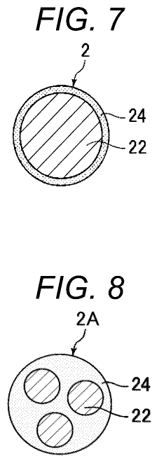

FIG. 7 is a cross-sectional view showing the magnetic bead 2 according to the embodiment. The magnetic bead 2 shown in FIG. 7 includes a magnetic metal particle 22 and a coating layer 24. A metal powder having magnetism is used as the magnetic metal particle 22. Therefore, the magnetic metal particle 22 has a high magnetization in a magnetic field and contributes to an improvement in a movement speed of the magnetic bead 2. Accordingly, a time required for the magnetic separation can be shortened.

Since the coating layer 24 contains an inorganic oxide, the coating layer 24 has a binding affinity with the biological material. Further, since the coating layer 24 contains the inorganic oxide, the coating layer 24 has a high function of protecting the magnetic metal particle 22 from oxidation, corrosion, and the like. Therefore, elution of metal ions in the magnetic bead 2 can be prevented.

In the magnetic separation operation, a particle group that is an aggregate of such a magnetic bead 2 is used. In the specification, the magnetic bead 2 refers to the particle group or one particle constituting the particle group.

### 2.1. Characteristics of Magnetic Bead

### 2.1.1. Metal Ion Concentration of Supernatant Generated by Hydrochloric Acid Immersion Test

When the magnetic bead 2 is subjected to a predetermined hydrochloric acid immersion test to be described later, a generated supernatant has a metal ion concentration of 0.01 ppm or more and 5.00 ppm or less in terms of mass ratio. Since the metal ion concentration of such a magnetic bead 2 is optimized, it is possible to prevent extraction of the biological material from being inhibited due to an excessively high metal ion concentration. Therefore, since the metal ion concentration of the supernatant generated in the hydrochloric acid immersion test is within the above-described range, the magnetic bead 2 that has high biological material extraction efficiency is obtained.

The predetermined hydrochloric acid immersion test includes the following operations (1) to (3).
(1) A first mixture is prepared by mixing 0.1 g of ethylenediaminetetraacetic acid (EDTA), 5 mL of pure water, and 100 mg of the magnetic bead 2.
(2) The first mixture and 5 mL of hydrochloric acid having a concentration of 10 mM are mixed to obtain a second mixture, and then the second mixture is stirred with a vortex mixer for 30 minutes.
(3) The stirred second mixture is allowed to stand for one day, then a supernatant is collected, and a metal ion concentration of the supernatant is measured by high-frequency inductively coupled plasma (ICP) emission spectroscopy.

The metal ion concentration of the supernatant can be measured by subjecting the magnetic bead 2 to the hydrochloric acid immersion test including such operations (1) to (3). The measured metal ion concentration of the supernatant is, as described above, 0.01 ppm or more and 5.00 ppm or less, preferably 0.10 ppm or more and 4.00 ppm or less, and more preferably 0.50 ppm or more and 3.00 ppm or less in terms of mass ratio.

When the metal ion concentration of the supernatant exceeds the upper limit value, corrosion resistance of the magnetic bead 2 is low, and therefore, for example, when the biological material is extracted in the dissolution and adsorption step S102, a large amount of metal ions are eluted. The metal ions form a complex with a component contained in the lysis and bindin buffer, and inhibit adsorption of the biological material to the magnetic bead 2. On the other hand, in order for the metal ion concentration of the supernatant to be lower than the lower limit value, it is necessary to increase the corrosion resistance of the magnetic bead 2, and there is a concern that the coating layer 24 needs to be particularly thickened or magnetic properties of the magnetic metal particle 22 deteriorate. In addition, it is found that the adsorbed biological material is less likely to elute when the metal ion concentration is less than the lower limit value. In this case, a yield of the biological material decreases.

Therefore, the metal ion concentration of the supernatant within the above-described range is useful from the viewpoint of improving the biological material extraction efficiency of the magnetic bead 2.

The metal ion concentration of the supernatant can be adjusted according to composition and a metal structure of the magnetic metal particle 22, a thickness and composition of the coating layer 24, and the like. For example, the metal ion concentration of the supernatant can be reduced by increasing the thickness of the coating layer 24.

### 2.1.2. Magnetic Properties

A saturation magnetization of the magnetic bead 2 is preferably 50 emu/g or more, more preferably 80 emu/g or more, and still more preferably 100 emu/g or more. The saturation magnetization is a magnetization value in a case where a magnetization exhibited by a magnetic material when a sufficiently large magnetic field is externally applied is constant regardless of the magnetic field. When the saturation magnetization is within the above-described range, a function as a magnetic material can be sufficiently exhibited. Specifically, since a movement speed of the magnetic bead 2 in a magnetic field can be increased, a time required for magnetic separation can be shortened. The saturation magnetization of the magnetic bead 2 affects an adsorption force when the magnetic bead 2 is fixed by an external magnetic field. When the saturation magnetization is within the above-described range, a sufficiently high adsorption force can be obtained, and therefore, when the liquid 3 is discharged in a state in which the magnetic bead 2 is fixed, the magnetic bead 2 can be prevented from being discharged together with the liquid 3. Accordingly, it is possible to prevent a decrease in a biological material yield due to a decrease in the number of the magnetic beads 2.

An upper limit value of the saturation magnetization of the magnetic bead 2 is not particularly limited, and is preferably 220 emu/g or less from the viewpoint of ease of selection of a material suitable for a balance between performance and cost.

The saturation magnetization of the magnetic bead 2 can be measured by a vibrating sample magnetometer (VSM) or the like. As the vibrating sample magnetometer, for example, TM-VSM1230-MHHL manufactured by Tamagawa Seisakusyo Co., Ltd. may be used. A maximum applied magnetic field when measuring the saturation magnetization is, for example, 0.5 T or more.

A coercive force Hc of the magnetic bead 2 is preferably 1500 A/m or less, more preferably 800 A/m or less, still more preferably 400 A/m or less, and particularly preferably 100 A/m or less. The coercive force Hc refers to a value of an external magnetic field in an opposite direction required to return a magnetized magnetic material to an unmagnetized state. That is, the coercive force Hc means a resistance force against an external magnetic field. As the coercive force Hc of the magnetic bead 2 decreases, the magnetic bead 2 is less likely to aggregate even when being switched from a state in which a magnetic field is applied to a state in which no magnetic field is applied, and the magnetic bead 2 can be uniformly dispersed in a dispersion liquid. Further, even when the switching of magnetic field application is repeated, since redispersibility of the magnetic bead 2 is improved as the coercive force Hc decreases, aggregation of the magnetic bead 2 can be further prevented. A lower limit value of the coercive force Hc of the magnetic bead 2 is not particularly limited, and is preferably 5 A/m or more from the viewpoint of ease of selection of a material suitable for a balance between performance and cost.

The coercive force Hc of the magnetic bead 2 can be measured by a vibrating sample magnetometer or the like in the same manner as the saturation magnetization described above. A maximum applied magnetic field when measuring the coercive force Hc is, for example, 15 kOe.

A relative permeability of the magnetic bead 2 is preferably 5 or more. When the relative permeability of the magnetic bead 2 is less than the lower limit value, responsiveness of the magnetic bead 2 decreases, and the time required for magnetic separation may be longer. An upper limit value of the relative permeability of the magnetic bead 2 is not particularly limited, and since the magnetic bead 2 is in a powder form, the relative permeability is usually substantially 100 or less due to an influence of a demagnetizing field.

### 2.1.3. Powder Characteristics

An average particle diameter D50 of the magnetic bead 2 is preferably 0.5 μm or more and 15 μm or less, more preferably 1 μm or more and 10 μm or less, still more preferably 2 μm or more and 8 μm or less, and particularly preferably 2 μm or more and 6 μm or less. When the average particle diameter D50 of the magnetic bead 2 is within the above-described range, a specific surface area of the magnetic bead 2 can be sufficiently large, and an attractive force and an adsorption force suitable for magnetic separation can be generated in the magnetic bead 2. In addition, aggregation of the magnetic bead 2 can be prevented, and dispersibility can be improved. When the average particle diameter D50 of the magnetic bead 2 is less than the lower limit value, a magnetization value of the magnetic bead 2 is small, the magnetic bead 2 is likely to aggregate, and as a result, the biological material extraction efficiency may decrease. In addition, the movement speed of the magnetic bead 2 may decrease, and the time required for magnetic separation may increase. On the other hand, when the average particle diameter D50 of the magnetic bead 2 exceeds the upper limit value, since the specific surface area of the magnetic bead 2 is small, a sufficient amount of the biological material cannot be adsorbed, and an extraction amount 41 the biological material may decrease. In addition, the magnetic bead 2 is likely to settle, the number of the magnetic beads 2 that can contribute to the extraction of the biological material may decrease, and the biological material extraction efficiency may decrease.

The average particle diameter D50 of the magnetic bead 2 can be obtained from a cumulative distribution curve obtained from a volume-based particle size distribution measured by a laser diffraction and dispersion method. Specifically, in the cumulative distribution curve, a particle diameter (median diameter) where a cumulative value is 50% from a small diameter side is the average particle diameter D50 of the magnetic bead 2. Examples of an apparatus for measuring the particle size distribution by the laser diffraction and dispersion method include MT3300 series manufactured by MicrotracBEL. The method is not limited to the laser diffraction and dispersion method, and a method such as image analysis may be used.

A 90% particle diameter of the magnetic bead 2 is defined as D90. In the magnetic bead 2, a ratio D90/D50 of the 90% particle diameter D90 to the average particle diameter D50 is preferably 3.00 or less, more preferably 2.00 or less, and still more preferably 1.75 or less. Accordingly, since a content of coarse particles is low, it is possible to prevent the coarse particles from attracting relatively small surrounding particles and aggregating to form an aggregate. When an aggregate is generated, the aggregate settles due to own weight, which may cause a decrease in extraction efficiency and an increase in an test time of the biological material. Therefore, when the ratio D90/D50 is within the above-described range, occurrence of such a problem can be prevented. When the ratio D90/D50 exceeds the upper limit value, the content of coarse particles is high, and therefore, even when the application of the external magnetic field is turned off, the dispersibility of the magnetic bead 2 decreases, and aggregation is likely to occur.

The 90% particle diameter D90 of the magnetic bead 2 can be obtained from a cumulative distribution curve obtained from a volume-based particle size distribution measured by a laser diffraction and dispersion method. Specifically, in the cumulative distribution curve, a particle diameter where a cumulative value is 90% from a small diameter side is the 90% particle diameter D90 of the magnetic bead 2.

### 2.2. Configuration of Magnetic Bead

As described above, the magnetic bead 2 shown in FIG. 7 includes the magnetic metal particle 22 and the coating layer 24. Hereinafter, each part will be described.

### 2.2.1. Magnetic Metal Particle

The magnetic metal particle 22 is a metal particle having magnetism, and preferably contains at least one of Fe, Co, and Ni as an element. Accordingly, the magnetic bead 2 that has a high magnetization derived from the magnetic metal is obtained.

In particular, from the viewpoint of obtaining a high saturation magnetization, the composition of the magnetic metal particle 22 is preferably an alloy containing Fe as a main component (Fe-based alloy). Specifically, an atomic ratio of Fe is more preferably 50% or more, and still more preferably 70% or more. Examples of the Fe-based alloy include a Fe—Co alloy, a Fe—Ni alloy, a Fe—Co—Ni alloy, and compounds containing Fe, Co, and Ni. From the viewpoint of obtaining high magnetization, carbonyl iron powder made of substantially 100 mass % of Fe may be used as the magnetic metal particle 22. Using such a Fe-based alloy, it is possible to obtain the magnetic metal particle 22 having a high saturation magnetization and a high permeability even when a particle diameter is small. Accordingly, it is possible to obtain the magnetic bead 2 whose movement speed due to the action of the external magnetic field is high and whose attractive force when captured by the external magnetic field is large. As a result, the time required for magnetic separation can be shortened, and the magnetic bead 2 can be prevented from being mixed into the elution buffer and becoming a contaminants.

The Fe-based alloy may contain one or two or more selected from the group consisting of Cr, Nb, Cu, Al, Mn, Mo, Si, Sn, B, C, P, Ti, and Zr according to target properties, in addition to an element exhibiting strong magnetism alone like Fe described above. Si is a main element in an alloy powder and is also an element that promotes amorphization.

The Fe-based alloy may contain an impurity as long as an effect of the magnetic metal particle 22 is not impaired. The impurity in the embodiment is an element that is unintentionally mixed during production of a raw material of the magnetic metal particle 22 or the magnetic bead 2. The impurity is not particularly limited, and examples thereof include O, N, S, Na, Mg, and K.

An example of the Fe-based alloy is an alloy having a Si content of preferably 1.0 atomic % or more and 30.0 atomic % or less, more preferably 1.5 atomic % or more and 14.0 atomic % or less, and still more preferably 2.0 atomic % or more and 7.0 atomic % or less. Since such an alloy has a high permeability, the saturation magnetization tends to be high.

The Fe-based alloy may contain at least one of B (boron) having a content of preferably 5.0 atomic % or more and 16.0 atomic % or less, more preferably 8.0 atomic % or more and 13.0 atomic % or less, and C (carbon) having a content of preferably 0.5 atomic % or more and 5.0 atomic % or less, more preferably 1.5 atomic % or more and 4.0 atomic % or less. These elements are elements that promote amorphization, and contribute to stably forming an amorphous structure or a nanocrystal structure in the magnetic metal particle 22. Accordingly, the magnetic metal particle 22 that has a low coercive force is obtained.

Further, the Fe-based alloy preferably contains chromium (Cr) having a content of 1.0 atomic % or more and 8.0 atomic % or less, more preferably 1.5 atomic % or more and 5.0 atomic % or less. Accordingly, corrosion resistance of the magnetic metal particle 22 can be improved. As a result, the magnetic metal particle 22 in which elution of metal ions is reduced is obtained.

A content of impurities is preferably 1.0 atomic % or less in total. At such a level, the effect of the magnetic metal particle 22 is not impaired even when impurities are contained.

The elements and the composition of the magnetic metal particle 22 can be specified by an ICP emission analysis method defined in JIS G 1258:2014, a spark emission analysis method defined in JIS G 1253:2002, or the like. When the magnetic metal particle 22 is covered with the coating layer 24, a measurement can be performed by the above-described methods after the coating layer 24 is removed by a chemical or physical method. When it is difficult to remove the coating layer 24, for example, analysis can be performed, after cutting the magnetic bead 2, on a portion of the magnetic metal particle 22 that is a core with an analysis apparatus such as an electron probe micro analyzer (EPMA) or energy dispersive X-ray spectroscopy (EDX).

A Vickers hardness of the magnetic metal particle 22 is preferably 100 or more, more preferably 300 or more, and still more preferably 800 or more. A method of measuring the hardness of the magnetic metal particle 22 is, for example, as follows. A plurality of particles of the magnetic metal particles 22 are taken out, embedded in a resin to prepare a resin-embedded sample, and then a cross-section of each magnetic metal particle 22 is exposed at a surface of the resin-embedded sample through grinding and polishing. The cross-section is subjected to indentation with a micro Vickers tester, a nanoindenter, or the like, and a hardness is measured from a size of the indentation.

When the Vickers hardness of the magnetic metal particle 22 is less than the lower limit value, the magnetic metal particle 22 may be plastically deformed by an impact when the magnetic bead 2 undergoes collision. When plastic deformation occurs, the coating layer 24 may be peeled off or detached. An upper limit value of the Vickers hardness is not particularly limited, and is preferably 3000 or less from the viewpoint of ease of selection of a material suitable for a balance of performance and cost.

A main metal structure constituting the magnetic metal particle 22 may take various forms such as a crystal structure, an amorphous structure, and a nanocrystal structure. An amorphous structure refers to a non-crystal structure in which no crystal is present, and a nanocrystal refers to a structure mainly formed of fine crystals having a crystal grain diameter of 100 nm or less. The amorphous structure and the nanocrystal structure impart a high hardness to the magnetic metal particle 22. When the structure is the amorphous structure or the nanocrystal structure, the coercive force Hc of the magnetic bead 2 has a particularly low value, which contributes to improvement in the redispersibility of the magnetic bead 2. A volume fraction of the magnetic metal particle 22 having an amorphous structure or a nanocrystal structure is preferably 40% or more, and more preferably 60% or more. The volume fraction is obtained from a result of crystal structure analysis by X-ray diffraction. Each of the crystal structure, the amorphous structure, and the nanocrystal structure may exist alone, or two or more thereof may co-exist.

The metal structure of the magnetic metal particle 22 can also be identified by a crystal structure analysis by an X-ray diffraction method on the magnetic metal particle 22. Alternatively, the metal structure can be specified by analyzing a structure observation image or a diffraction pattern obtained with a transmission electron microscope (TEM) from a cut-out sample. For example, in the case of the amorphous structure, a diffraction peak derived from a metal crystal of an α-Fe phase or the like is not observed in peak analysis of the X-ray diffraction method. In the case of the amorphous structure, a so-called halo pattern is formed in an electron diffraction pattern by TEM, and formation of a spot due to a crystal is not observed. The nanocrystal structure is formed of a crystal structure having a grain size of, for example, 100 nm or less, and can be checked from a TEM observation image. More accurately, an average grain diameter can be calculated by image processing or the like from a plurality of TEM structure observation images in which there are a plurality of crystals. In addition, the crystal grain diameter can be estimated by a Scherrer method based on a diffraction peak of a target crystal phase obtained by an X-ray diffraction method. Further, for a crystal structure having a large grain diameter, the crystal grain diameter can be measured by a method such as observing a cross-section with an optical microscope or a scanning electron microscope (SEM).

In order to obtain the amorphous structure and the nanocrystal structure, it is effective to increase a cooling rate at the time of cooling after a molten raw material is pulverized when producing the magnetic metal particle 22. Ease of forming the amorphous structure and the nanocrystal structure also depends on an alloy composition. As a specific alloy system suitable for forming the amorphous structure and the nanocrystal structure, a composition in which one or two or more selected from the group consisting of Cr, Si, B, C, P, Nb, and Cu are added to Fe is preferable.

The magnetic metal particle 22 is produced by a method according to a general metal powder production method. Examples of the production method include a melting process in which a metal is melted, solidified, and powdered, a chemical process in which a powder is produced by a reduction method or a carbonyl method, and a mechanical process in which a metal having a larger shape such as an ingot is mechanically pulverized to obtain a powder. Among these, the melting process is suitable for producing the magnetic metal particle 22.

Among production methods based on the melting process, a representative production method is an atomization method. The atomization method causes a molten metal to collide with a fluid (liquid or gas) injected at a high speed, be rapidly quenched and solidified to form a powder, and is classified into a water atomization method, a high-pressure water atomization method, a rotary water jet atomization method, a gas atomization method, and the like depending on a difference in a type of a cooling medium or an apparatus configuration. According to the atomization method, the magnetic metal particle 22 can be efficiently produced. Further, in the high-pressure water atomization method, the rotary water jet atomization method, and the gas atomization method, a particle shape of the metal powder is closer to a spherical shape due to an action of surface tension.

### 2.2.2. Coating Layer

As shown in FIG. 7, the coating layer 24 covers a particle surface of the magnetic metal particle 22. The coating layer 24 may be formed at at least a part of the particle surface of the magnetic metal particle 22, and preferably covers the entire particle surface.

The coating layer 24 covers the particle surface of the magnetic metal particle 22 and contains an inorganic oxide. Examples of the inorganic oxide include a silicon oxide, a magnesium oxide, a calcium oxide, an aluminum oxide, a titanium oxide, a zirconium oxide, a boron oxide, and an yttrium oxide, and a mixture of one or two or more thereof may be used. Since the coating layer 24 containing such an inorganic oxide is porous, specific surface area is increased. Accordingly, an adsorption amount of the biological material to the coating layer 24 can be increased.

The inorganic oxide is particularly preferably a silicon oxide. The silicon oxide specifically adsorbs the biological material such as a nucleic acid in the lysis and bindin buffer, thereby enabling efficient extraction and collection of the biological material. Further, since the silicon oxide is chemically stable, oxidation and corrosion of the magnetic metal particle 22 can be particularly prevented, and the corrosion resistance of the magnetic bead 2 can be particularly improved.

The silicon oxide is represented by a composition formula of SiOₓ (0<x≤2), and is preferably SiO₂. The silicon oxide may form a composite oxide or a composite with one or two or more selected from the group consisting of Al, Ti, V, Nb, Cr, Mn, Sn, and Zr.

The inorganic oxide may contain a substance (impurity) other than the inorganic oxide within a range in which the above-described effects are not impaired, for example, at a ratio of preferably 50 mass % or less, more preferably 10 mass % or less of the above-described inorganic oxide. When the silicon oxide is used as the inorganic oxide, examples of the impurity include C, N, and P.

A composition of the inorganic oxide can be checked by, for example, EDX analysis or Auger electron spectroscopy.

An average thickness of the coating layer 24 is preferably 15 nm or more, more preferably 20 nm or more, and still more preferably 30 nm or more. Accordingly, even when the magnetic bead 2 collides with each other or collides with the inner wall of the container or the like, the coating layer 24 can be prevented from being broken or peeled off. As a result, elution of metal ions and the like due to exposure of the magnetic metal particle 22 can be prevented. Meanwhile, the average thickness of the coating layer 24 is preferably 500 nm or less, more preferably 200 nm or less, and still more preferably 150 nm or less. Accordingly, a decrease in a magnetization per volume of the magnetic bead 2 can be prevented, and a decrease in the movement speed of the magnetic bead 2 can be prevented.

A ratio t/D50 of t to D50, in which t is the average thickness of the coating layer 24 and D50 is the average particle diameter of the magnetic bead 2, is preferably 0.0001 or more and 0.05 or less, more preferably 0.001 or more and 0.04 or less, and still more preferably 0.005 or more and 0.03 or less. When t/D50 is less than the lower limit value, a ratio of the thickness of the coating layer 24 to the size of the magnetic metal particle 22 is excessively small, and thus the coating layer 24 may be broken or peeled off when the magnetic bead 2 collides with each other or the magnetic bead 2 collides with the inner wall of the container 1 or the like. Therefore, an amount of the biological material adsorbed and extracted by the surface of the coating layer 24 decreases, and the extraction efficiency may decrease. When there are fragments of the peeled coating layer 24 or the magnetic metal particle 22 in an extracted liquid, the fragments may be mixed as a contamination at the same time when the biological material is extracted. Further, the magnetic metal particle 22 may be exposed due to breaking and peeling of the coating layer 24, and elution of metal ions or the like may occur when the magnetic metal particle 22 comes into contact with an acidic solution or the like, resulting in a decrease in the biological material extraction efficiency. On the other hand, when t/D50 exceeds the upper limit value, a volume ratio of the coating layer 24 to an entire volume of the magnetic bead 2 is large, and a magnetization per volume of the magnetic bead 2 may decrease. Accordingly, the movement speed when the external magnetic field acts on the magnetic bead 2 decreases, and the time required for magnetic separation may increase.

The thickness of the coating layer 24 can be measured from, for example, a cross-sectional observation image of the magnetic bead 2 observed by a transmission electron microscope or a scanning electron microscope. The average thickness t of the coating layer 24 can be calculated by acquiring a plurality of observation images and averaging measured values from image processing or the like. For example, the average thickness t is a value obtained by measuring the thickness of the coating layer 24 at five or more positions for one magnetic bead 2, obtaining an average value thereof, and then averaging the average value for ten or more magnetic beads 2. For example, intensities of a Si—K characteristic X-ray and a Fe-L characteristic X-ray may be compared using an analysis apparatus such as energy dispersive X-ray spectroscopy (EDX), and the thickness of the coating layer 24 may be calculated based on a comparison result. That is, as will be described later, when the coating layer 24 contains silicon and the magnetic metal particle 22 includes a Fe-based alloy, an intensity ratio of a Si—K characteristic X-ray derived from the coating layer 24 to a sum of a Fe-L characteristic X-ray derived from the magnetic metal particle 22 and a Si—K characteristic X-ray derived from the coating layer 24 can be converted into the thickness of the coating layer 24.

Examples of a method for forming the coating layer 24 include a wet formation method such as a sol-gel method and a dry formation method such as a vapor-phase deposition method. Among these, a Stöber method, which is a type of the sol-gel method, or an atomic layer deposition (ALD) method can be preferably used. The Stöber method is a method of forming monodisperse particles by hydrolysis of a metal alkoxide. For example, when the coating layer 24 is formed of the silicon oxide, the silicon oxide can be produced by a hydrolysis reaction of a silicon alkoxide. Before the coating layer 24 is formed, a base thereof, for example, a surface of a particle of the magnetic metal particle 22 may be subjected to a washing treatment using water or an organic solvent.

The coating layer 24 may cover a surface of one magnetic metal particle 22, or may cover a plurality of magnetic metal particles 22 together.

FIG. 8 is a cross-sectional view showing a modification of the magnetic bead 2 in FIG. 7.

A magnetic bead 2A shown in FIG. 8 contains a plurality of magnetic metal particles 22. The coating layer 24 covers the plurality of magnetic metal particles 22. With such a magnetic bead 2A, the same effects as those of the magnetic bead 2 shown in FIG. 7 can still be obtained.

The number of the magnetic metal particles 22 of the magnetic bead 2A is not particularly limited, and is 2 or more and 100 or less.

### 3. Magnetic Bead Dispersion Liquid

Next, the magnetic bead dispersion liquid 4 according to the embodiment will be described. As described above, the magnetic bead dispersion liquid 4 shown in FIG. 2 contains the magnetic bead 2 and the dispersion medium 40.

Examples of the dispersion medium 40 include polar organic solvents such as water, saltwater, and alcohols, and aqueous solutions thereof. Examples of water include sterilized water and pure water. Examples of alcohols include ethanol and isopropyl alcohol.

A concentration of the magnetic bead 2 in the magnetic bead dispersion liquid 4 is not particularly limited as long as sufficient uniformity can be ensured by stirring the magnetic bead dispersion liquid 4.

A surfactant may be added for the purpose of improving the dispersibility of the magnetic bead 2 in the magnetic bead dispersion liquid 4. Examples of the surfactant include a non-ionic surfactant, a cationic surfactant, an anionic surfactant, and an amphoteric surfactant.

Examples of the non-ionic surfactant include Triton (registered trademark) series surfactants such as Triton-X, Tween (registered trademark) series surfactants such as Tween 20, and acyl sorbitan. Examples of the cationic surfactant include dodecyltrimethylammonium bromide, dodecyltrimethylammonium chloride, and cetyltrimethylammonium bromide. Examples of the anionic surfactant include sodium dodecyl sulfate (SDS), sodium N-lauroylsarcosinate, sodium cholate, sodium lauryl sulfate, and sarcosine. An example of the amphoteric surfactant is phosphatidylethanolamine. These surfactants are each used alone or two or more thereof are used in combination.

A content of the surfactant in the magnetic bead dispersion liquid 4 is preferably equal to or higher than a critical micelle concentration of the surfactant. The critical micelle concentration is also referred to as cmc and refers to a concentration where molecules of a surfactant dispersed in a liquid aggregate to form a micelle. When the content of the surfactant is equal to or higher than the critical micelle concentration, the surfactant is likely to form a layer around the magnetic bead 2. Accordingly, an effect of preventing aggregation of the magnetic bead 2 can be further improved.

The content of the surfactant is not limited to be equal to or higher than the critical micelle concentration, and may be less than the critical micelle concentration. For example, the content of the surfactant in the magnetic bead dispersion liquid 4 is preferably 0.05 mass % or more and 3.0 mass % or less regardless of the critical micelle concentration.

Further, it is preferable to add a preservative in order to impart long-term storage stability and a preservation effect to the magnetic bead dispersion liquid 4. An example of the preservative is sodium azide. A concentration of the preservative added is preferably 0.02 mass % or more and less than 0.1 mass % of the magnetic bead dispersion liquid 4. When the content is less than 0.02 mass %, a sufficient effect for long-term storage stability and preservation may not be obtained, and when the content is equal to or higher than 0.1 mass %, problems such as lowering the biological material extraction efficiency may occur.

A buffer may be added for the purpose of pH adjustment. An example of the buffer is Tris buffer.

### 4. Effects of Embodiment

As described above, the magnetic bead 2 according to the embodiment includes the magnetic metal particle 22 and the coating layer 24. The coating layer 24 covers the surface of the magnetic metal particle 22, contains the inorganic oxide, and has an average thickness of 15 nm or more. The metal ion concentration of the supernatant obtained by subjecting the magnetic bead 2 to the following operations is 0.01 ppm or more and 5.00 ppm or less. The following operations include the operation of mixing 0.1 g of ethylenediaminetetraacetic acid (EDTA), 5 mL of pure water, and 100 mg of the magnetic bead 2 to prepare the first mixture, the operation of mixing the first mixture with 5 mL of hydrochloric acid having a concentration of 10 mM to obtain the second mixture, and then stirring the second mixture with a vortex mixer for 30 minutes, and the operation of allowing the stirred second mixture to stand for one day, then collecting the supernatant, and measuring the metal ion concentration of the supernatant by high-frequency inductively coupled plasma (ICP) emission spectroscopy.

According to such a configuration, since the magnetic metal particle 22 has a high magnetization derived from the magnetic metal, it is possible to obtain the magnetic bead 2 that has a high movement speed under an action of an external magnetic field and can rapidly perform magnetic separation. Since the metal ion concentration of the supernatant obtained by the above operations is optimized, the magnetic bead 2 that has high biological material extraction efficiency is obtained.

The inorganic oxide is preferably a silicon oxide.

Since the silicon oxide is chemically stable, oxidation and corrosion of the magnetic metal particle 22 can be particularly prevented, and the corrosion resistance of the magnetic bead 2 can be particularly improved. The silicon oxide specifically adsorbs the biological material such as a nucleic acid in the lysis and bindin buffer, thereby enabling efficient extraction and collection of the biological material.

The magnetic metal particle 22 is preferably made of a Fe-based alloy.

According to such a configuration, it is possible to obtain the magnetic metal particle 22 having a high saturation magnetization and a high permeability even when the particle diameter is small. Accordingly, it is possible to obtain the magnetic bead 2 whose movement speed due to the action of the external magnetic field is high and whose attractive force when captured by the external magnetic field is large. As a result, the time required for magnetic separation can be shortened, and the magnetic bead 2 can be prevented from being mixed into the elution buffer and becoming a contaminant.

The Fe-based alloy preferably contains Fe as a main component and one or two or more selected from the group consisting of Cr, Si, B, C, P, Nb, and Cu.

Accordingly, the Fe-based alloy that is likely to form an amorphous structure or a nanocrystal structure is obtained. As a result, the magnetic bead 2 that has a low coercive force and excellent redispersibility can be obtained.

The magnetic metal particle 22 preferably includes an amorphous structure.

According to such a configuration, the magnetic bead 2 that has a low coercive force and excellent redispersibility can be obtained.

In a cumulative distribution curve obtained from a volume-based particle size distribution, the particle diameter D50 where a cumulative value from a small diameter side is 50% is preferably 0.5 μm or more and 15 μm or less.

According to such a configuration, the specific surface area of the magnetic bead 2 can be sufficiently large, and an attractive force and an adsorption force suitable for magnetic separation can be generated in the magnetic bead 2. In addition, aggregation of the magnetic bead 2 can be prevented, and dispersibility can be improved.

The ratio t/D50 of the average thickness t of the coating layer 24 to the particle diameter D50 is preferably 0.0001 or more and 0.05 or less.

With such a configuration, peeling or the like of the coating layer 24 due to collision of the magnetic bead 2 can be prevented. In addition, elution of metal ions and the like can be prevented. Further, a decrease in the magnetization per volume of the magnetic bead 2 can be prevented. As a result, magnetic separation can be performed more rapidly, and the magnetic bead 2 having higher biological material extraction efficiency is obtained.

The magnetic bead dispersion liquid 4 as the magnetic bead reagent according to the embodiment contains the magnetic bead 2 and the dispersion medium 40 in which the magnetic bead 2 is dispersed.

With such a configuration, since the magnetic metal particle 22 has a high magnetization derived from the magnetic metal, it is possible to obtain the magnetic bead dispersion liquid 4 that has a high movement speed under the action of the external magnetic field and can rapidly perform magnetic separation. Since the metal ion concentration of the supernatant obtained by the above operations is optimized, the magnetic bead dispersion liquid 4 that has high biological material extraction efficiency is obtained.

The magnetic bead and the magnetic bead reagent according to the disclosure are described above based on the shown embodiment, but the disclosure is not limited thereto. For example, the magnetic bead and the magnetic bead reagent according to the disclosure may be obtained by adding any component to the embodiment.

### EXAMPLES

Next, specific examples of the disclosure will be described.

### 5. Preparation of Magnetic Bead

First, a magnetic metal particle made of a soft magnetic material having the composition shown in Table 1 was prepared by a high-pressure water atomization method. A metal structure of the obtained magnetic metal particle was analyzed by an X-ray diffraction method. The metal structure specified by the analysis is shown in Table 1.

TABLE 1

| Soft magnetic | | Composition formula |
|---|---|---|
| material | Metal structure | (atomic ratio) |
| 1 | Non-crystalline | Fe₈₁Si₅B₁₂C₂ |
| 2 | Non-crystalline | Fe₇₃Si₁₁Cr₂B₁₁C₃ |
| 3 | Non-crystalline | Fe_{73.5}Si_{13.5}CU₁B₉Nb₃ |
| 4 | Crystalline | Fe_{88.6}Si_{6.7}Cr_{4.7} |

Next, a silicon oxide (SiO₂) was formed into a layer at a surface of the magnetic metal particle by a Stöber method to obtain a coating layer. In the Stöber method, first, 100 g of a magnetic metal powder was dispersed in 950 mL of ethanol and mixed, and the mixed liquid was stirred for 20 minutes by an ultrasonic application apparatus. After the stirring, a mixed solution of 30 mL of pure water and 180 mL of ammonia water was added, followed by stirring for 10 minutes. Thereafter, a mixed liquid of tetraethoxysilane (TEOS) and 100 mL of ethanol was further added and stirred to form the coating layer at the surface of the magnetic metal particle. A formation time in this case was 30 minutes, and the formation time was changed according to a thickness to be formed. Thereafter, the obtained coating layer was washed with ethanol and acetone, respectively. After the washing, the resultant was dried at 65° C. for 30 minutes and further heated at 200° C. for 90 minutes. Accordingly, magnetic beads in samples No. 1 to No. 32 shown in Tables 2 to 6 were obtained.

In preparation of a magnetic bead of sample No. 29, tetraethoxytitanium was used as a metal alkoxide.

A saturation magnetization and a coercive force of the magnetic bead are shown in Tables 2 to 6.

### 6. Measurement of Metal Ion Concentration of Supernatant

Magnetic beads of each of samples No. 1 to No. 25 were subjected to the predetermined hydrochloric acid immersion test described above. Then, a metal ion concentration of a generated supernatant was measured. Measurement results are shown in Tables 2 to 5.

In Tables 2 to 5, magnetic beads corresponding to the disclosure are each indicated by “Example”, and magnetic beads not corresponding to the disclosure are each indicated by “Comparative Example”.

### 7. Preparation of Magnetic Bead Suspension for Evaluation

Magnetic beads of each of samples No. 26 to No. 32 were added to pure water containing iron sulfate to prepare a magnetic bead suspension. In the obtained magnetic bead suspension, an addition amount of iron sulfate was adjusted such that a metal ion concentration is a concentration shown in Table 6.

Since each of samples No. 26 to No. 32 is a magnetic bead suspension in which the metal ion concentration is intentionally adjusted by iron sulfate, these samples are each shown as “Reference Example” for evaluation in Table 6.

### 8. Evaluation of Magnetic Bead

### 8.1. Collection Amount and Yield of Nucleic Acid

First, human genomic DNA was prepared as a nucleic acid model, and lysozyme was prepared as a contaminant model.

Next, the magnetic beads of each of Examples and Comparative Examples were dispersed in pure water and stirred to prepare magnetic bead suspension. The magnetic bead suspension of each of Reference Examples (samples No. 26 to No. 32) was used as it is. A content of the magnetic bead in the magnetic bead suspension was 53.17 mass %.

Next, reagents other than the nucleic acid were left to stand to reach a room temperature, and then the reagents were charged into tubes in the following order.
Pure water: 10 μL
Nucleic acid dispersion liquid having a concentration of 0.1 μg/μL: 75 μL
Lysis and bindin buffer: 750 μL
Lysozyme aqueous solution having a concentration of 10 μg/μL: 15 μL
Magnetic bead suspension: 40 μL

Next, contents of the tube were stirred with a vortex mixer for 10 minutes, then the tube was set at a magnetic stand and allowed to stand for 30 seconds. When the magnetic bead was magnetically trapped, the supernatant was removed.

Next, 900 μL of a washing liquid was added to the tube, followed by stirring with a vortex mixer for 5 seconds and centrifugation. Thereafter, the tube was set at a magnetic stand and allowed to stand for 30 seconds. When the magnetic bead was magnetically trapped, the supernatant was removed. Thereafter, addition of the washing liquid, magnetic trapping, and removal of the supernatant were performed once again.

Next, 900 μL of 70% ethanol aqueous solution was added to the tube, followed by stirring with a vortex mixer for 5 seconds and centrifugation. Thereafter, the contents of the tube were suctioned with a pipette and returned once. Thereafter, the tube was set at a magnetic stand and allowed to stand for 30 seconds. When the magnetic bead was magnetically trapped, the supernatant was removed. Thereafter, addition of the ethanol aqueous solution, magnetic trapping, and removal of the supernatant were performed once again. Next, the tube was subjected to centrifugation, and the remaining supernatant was removed.

Next, 100 μL of pure water was added to the tube, followed by stirring with a vortex mixer for 10 minutes to elute the nucleic acid. Thereafter, the tube was set at a magnetic stand and allowed to stand for 30 seconds. When the magnetic bead was magnetically trapped, an elution buffer containing the nucleic acid was collected into another tube.

Next, the tube containing the collected elution buffer was set in a spectrophotometer, and a concentration of the nucleic acid in the elution buffer was determined based on an absorbance at a wavelength of 260 nm. Since a nucleotide base has absorption maximum near 260 nm, the absorbance at the wavelength of 260 nm was used to quantify the concentration of the nucleic acid. Then, a nucleic acid collection amount was calculated based on the obtained concentration. Calculation results are shown in Tables 2 to 6.

The calculated nucleic acid collection amount was evaluated in view of the following evaluation criteria. Evaluation results are shown in Tables 2 to 6 as relative evaluation results of a yield of the nucleic acid.
A: the nucleic acid collection amount was 35 ng/μL or more
B: the nucleic acid collection amount was 30 ng/μL or more and less than 35 ng/μL
C: the nucleic acid collection amount was 25 ng/μL or more and less than 30 ng/μL
D: the nucleic acid collection amount was less than 25 ng/μL

### 8.2. Redispersibility

First, a dispersion liquid was prepared by dispersing the magnetic beads of each of Examples and Comparative Examples in a PBS buffer aqueous solution to have a concentration of 30 mass %. The PBS buffer aqueous solution was a phosphate buffer containing NaCl having a concentration of 137 mmol/L, Na₂HPO₄ having a concentration of 8.1 mmol/L, KCl having a concentration of 2.7 mmol/L, and KH₂PO₄ having a concentration of 1.5 mmol/L.

Next, the prepared dispersion liquid was set in a laser diffraction and scattering particle diameter distribution measuring apparatus, and a volume-based average particle diameter was measured. A measured value of the obtained average particle diameter is taken as a value before separation of a target biological material and is taken as a reference value.

Next, a protein A as the target biological material was dispersed in pure water to prepare a specimen aqueous solution in a microtube. A concentration of the protein A was 50 μg/mL.

Next, the magnetic bead was dispersed in the specimen aqueous solution to have a concentration of 30 mass %. Accordingly, the target biological material was adsorbed to the magnetic bead.

Next, a magnet was brought close to a side surface of the microtube, a magnetic field was applied, and then a supernatant was removed. Then, the magnetic bead remaining in the microtube was collected.

Next, the collected magnetic bead was redispersed in a PBS buffer aqueous solution to prepare a redispersion liquid.

Next, the prepared redispersion liquid was set in the laser diffraction and scattering particle diameter distribution measuring apparatus, and a volume-based average particle diameter was measured. A measured value of the obtained average particle diameter is taken as a value after separation of the target biological material.

Next, a ratio of the value after separation to the reference value was calculated. As the ratio is closer to 1.0, redispersibility of the magnetic bead is more favorable. Subsequently, the calculated ratio was evaluated in view of the following evaluation criteria. Evaluation results are shown in Tables 2 to 5.
A: the ratio of the value after separation to the reference value is less than 1.1
B: the ratio of the value after separation to the reference value is 1.1 or more and less than 1.5
C: the ratio of the value after separation to the reference value is 1.5 or more and less than 2.0
D: the ratio of the value after separation to the reference value is 2.0 or more

### 8.3. Magnetic Separation Rate

First, a dispersion liquid was prepared by dispersing the magnetic beads of each of Examples and Comparative Examples in pure water at 25° C. to have a concentration of 0.1 mass %. Next, the dispersion liquid was charged in a spectroscopic cell and stirred with a vortex mixer. A stirring time was 1 minute. Next, the spectroscopic cell subjected to the stirring treatment was quickly set in a cell holder of a spectrophotometer. A magnet was attached to the cell holder in advance according to a position where the spectroscopic cell was disposed. The shortest distance between an outer wall of the spectroscopic cell set in the cell holder and the magnet was 2.0 mm, and a magnet having a surface magnetic flux density of 180 mT was used as the magnet.

Next, simultaneously with the start of standing of the spectroscopic cell, measurement of an absorbance at a wavelength of 550 nm in the spectroscopic cell was started. Then, a time until the measured absorbance was attenuated to 10% of an initial absorbance was measured, and a measurement result thereof was used as an evaluation index for evaluating a magnetic separation rate. The obtained evaluation index was evaluated in view of the following evaluation criteria. Evaluation results are shown in Tables 2 to 5.
A: the evaluation index of the magnetic separation rate is shorter than 20 seconds
B: the evaluation index of the magnetic separation rate is 20 seconds or longer and shorter than 40 seconds
C: the evaluation index of the magnetic separation rate is 40 seconds or longer and shorter than 60 seconds
D: the evaluation index of the magnetic separation rate is 60 seconds or longer

TABLE 2

| | | Configuration of magnetic bead | | | | | | | |
|---|---|---|---|---|---|---|---|---|---|
| | | Magnetic | | | | | | | |
| | | metal | | | | | | | |
| | | particle | | | | | | | |
| | | | Average | Coating film | | | Magnetic | | |
| | | Soft | particle | | Average | | properties | | |
| | | magnetic | diameter | Constituent | thickness | | Saturation | Coercive | |
| Sample | | material | D50 | material | t | t/D50 | magnetization | force | |
| No. | Classification | — | μm | — | nm | — | emu/g | A/m | Ce |
| 1 | Comparative | 1 | 5 | Silicon | 10 | 0.0020 | 151 | 24 | 0.3 |
| | Example | | | oxide | | | | | |
| 2 | Example | 1 | 5 | Silicon | 30 | 0.0060 | 151 | 24 | 0.3 |
| | | | | oxide | | | | | |
| 3 | Example | 1 | 5 | Silicon | 60 | 0.0120 | 151 | 24 | 0.3 |
| | | | | oxide | | | | | |
| 4 | Example | 1 | 5 | Silicon | 90 | 0.0180 | 151 | 24 | 0.3 |
| | | | | oxide | | | | | |
| 5 | Example | 1 | 5 | Silicon | 120 | 0.0240 | 151 | 24 | 0.3 |
| | | | | oxide | | | | | |
| 6 | Example | 1 | 5 | Silicon | 200 | 0.0400 | 151 | 24 | 0.3 |
| | | | | oxide | | | | | |

| | Configuration of magnetic bead | Evaluation result of magnetic bead | | | |
|---|---|---|---|---|---|
| | Metal | Nucleic | Relative | | |
| | ion | acid | evaluation | | Magnetic |
| | concentration | collection | of | | separation |
| Sample | of supernatant | amount | yield | Redispersibility | rate |
| No. | ppm | ng/μL | — | — | — |
| 1 | 5.6 | 20.5 | D | B | A |
| 2 | 1.5 | 30.6 | B | A | A |
| 3 | 0.5 | 34.5 | B | A | A |
| 4 | 0.1 | 32.2 | B | A | A |
| 5 | 0.1 | 35.6 | A | A | B |
| 6 | 0.1 | 36.5 | A | A | C |

TABLE 3

| | | Configuration of magnetic bead | | | | | | | |
|---|---|---|---|---|---|---|---|---|---|
| | | Magnetic | | | | | | | |
| | | metal | | | | | | | |
| | | particle | | | | | | | |
| | | | Average | Coating film | | | Magnetic | | |
| | | Soft | particle | | Average | | properties | | |
| | | magnetic | diameter | Constituent | thickness | | Saturation | Coercive | |
| Sample | | material | D50 | material | t | t/D50 | magnetization | force | |
| No. | Classification | — | μm | — | nm | — | emu/g | A/m | Ce |
| 7 | Comparative | 2 | 2 | Silicon | 5 | 0.0025 | 190 | 159 | 2 |
| | Example | | | oxide | | | | | |
| 8 | Comparative | 2 | 2 | Silicon | 10 | 0.0050 | 190 | 159 | 2 |
| | Example | | | oxide | | | | | |
| 9 | Example | 2 | 2 | Silicon | 20 | 0.0100 | 190 | 159 | 2 |
| | | | | oxide | | | | | |
| 10 | Example | 2 | 2 | Silicon | 30 | 0.0150 | 190 | 159 | 2 |
| | | | | oxide | | | | | |
| 11 | Example | 2 | 2 | Silicon | 45 | 0.0225 | 190 | 159 | 2 |
| | | | | oxide | | | | | |
| 12 | Example | 2 | 2 | Silicon | 60 | 0.0300 | 190 | 159 | 2 |
| | | | | oxide | | | | | |

| | Configuration of magnetic bead | Evaluation result of magnetic bead | | | |
|---|---|---|---|---|---|
| | Metal | Nucleic | Relative | | |
| | ion | acid | evaluation | | Magnetic |
| | concentration | collection | of | | separation |
| Sample | of supernatant | amount | yield | Redispersibility | rate |
| No. | ppm | ng/μL | — | — | — |
| 7 | 6.2 | 18.5 | D | C | A |
| 8 | 5.3 | 25.1 | C | B | A |
| 9 | 2.5 | 30.9 | B | A | A |
| 10 | 1.2 | 34.2 | B | A | A |
| 11 | 0.2 | 34.1 | B | A | A |
| 12 | 0.1 | 36.5 | A | A | A |

TABLE 4

| | | Configuration of magnetic bead | | | | | | | |
|---|---|---|---|---|---|---|---|---|---|
| | | Magnetic | | | | | | | |
| | | metal | | | | | | | |
| | | particle | | | | | | | |
| | | | Average | Coating film | | | Magnetic | | |
| | | Soft | particle | | Average | | properties | | |
| | | magnetic | diameter | Constituent | thickness | | Saturation | Coercive | |
| Sample | | material | D50 | material | t | t/D50 | magnetization | force | |
| No. | Classification | — | μm | — | nm | — | emu/g | A/m | Ce |
| 13 | Comparative | 3 | 5 | Silicon | 10 | 0.0020 | 119 | 80 | 1 |
| | Example | | | oxide | | | | | |
| 14 | Comparative | 3 | 5 | Silicon | 30 | 0.0060 | 119 | 80 | 1 |
| | Example | | | oxide | | | | | |
| 15 | Example | 3 | 5 | silicon | 60 | 0.0120 | 119 | 80 | 1 |
| | | | | oxide | | | | | |
| 16 | Example | 3 | 5 | silicon | 90 | 0.0180 | 119 | 80 | 1 |
| | | | | oxide | | | | | |
| 17 | Example | 3 | 5 | Silicon | 120 | 0.0240 | 119 | 80 | 1 |
| | | | | oxide | | | | | |
| 18 | Example | 3 | 5 | Silicon | 200 | 0.0400 | 119 | 80 | 1 |
| | | | | oxide | | | | | |

| | Configuration of magnetic bead | Evaluation result of magnetic bead | | | |
|---|---|---|---|---|---|
| | Metal | Nucleic | Relative | | |
| | ion | acid | evaluation | | Magnetic |
| | concentration | collection | of | | separation |
| Sample | of supernatant | amount | yield | Redispersibility | rate |
| No. | ppm | ng/μL | — | — | — |
| 13 | 8.4 | 15.2 | D | B | A |
| 14 | 6.4 | 25.5 | C | A | A |
| 15 | 2.1 | 30.1 | B | A | A |
| 16 | 1.1 | 33.3 | B | A | A |
| 17 | 0.3 | 37.2 | A | A | B |
| 18 | 0.1 | 35.4 | A | A | C |

TABLE 5
magnetic beag

| | | Configuration of magnetic bead | | | | | | | |
|---|---|---|---|---|---|---|---|---|---|
| | | Magnetic | | | | | | | |
| | | metal | | | | | | | |
| | | particle | | | | | | | |
| | | | Average | Coating film | | | Magnetic | | |
| | | Soft | particle | | Average | | properties | | |
| | | magnetic | diameter | Constituent | thickness | | Saturation | Coercive | |
| Sample | | material | D50 | material | t | t/D50 | magnetization | force | |
| No. | Classification | — | μm | — | nm | — | emu/g | A/m | Ce |
| 19 | Comparative | 4 | 8 | Silicon | 10 | 0.0013 | 185 | 797 | 10 |
| | Example | | | oxide | | | | | |
| 20 | Comparative | 4 | 8 | silicon | 30 | 0.0038 | 185 | 797 | 10 |
| | Example | | | oxide | | | | | |
| 21 | Example | 4 | 8 | Silicon | 60 | 0.0075 | 185 | 797 | 10 |
| | | | | oxide | | | | | |
| 22 | Example | 4 | 8 | Silicon | 60 | 0.0075 | 185 | 797 | 10 |
| | | | | oxide | | | | | |
| 23 | Example | 4 | 8 | Silicon | 90 | 0.0113 | 185 | 797 | 10 |
| | | | | oxide | | | | | |
| 24 | Example | 4 | 8 | Silicon | 120 | 0.0150 | 185 | 797 | 10 |
| | | | | oxide | | | | | |
| 25 | Example | 4 | 8 | Silicon | 200 | 0.0250 | 185 | 797 | 10 |
| | | | | oxide | | | | | |

| | Configuration of magnetic bead | Evaluation result of magnetic bead | | | |
|---|---|---|---|---|---|
| | Metal | | Relative | | |
| | ion | Nucleic | evaluation | | Magnet |
| | concentration | collection | of | | separation |
| Sample | of supernatant | amount | yield | Redispersibility | rate |
| No. | ppm | ng/μL | — | — | — |
| 19 | 12.3 | 13.6 | D | C | A |
| 20 | 7.8 | 22.5 | C | C | A |
| 21 | 3.6 | 30.1 | B | B | A |
| 22 | 3.3 | 30.3 | B | B | A |
| 23 | 2.1 | 30.5 | B | B | A |
| 24 | 1.5 | 32.8 | B | B | B |
| 25 | 0.3 | 30.2 | B | B | C |

TABLE 6

| | | Configuration of magnetic bead | | | | | | | |
|---|---|---|---|---|---|---|---|---|---|
| | | Magnetic | | | | | | | |
| | | metal | | | | | | | |
| | | particle | | | | | | | |
| | | | Average | | | | Magnetic | | |
| | | Soft | particle | Coating film | | | Properties | | |
| | | magnetic | diameter | Constituent | Average | | Saturation | Coercive | |
| Sample | | material | D50 | material | thickness | t/D50 | magnetization | force | |
| No. | Classification | — | μm | — | nm | — | emu/g | A/m | Ce |
| 26 | Reference | 1 | 12 | Silicon | 60 | 0.0050 | 144 | 32 | 0.4 |
| | Example | | | oxide | | | | | |
| 27 | Reference | 1 | 12 | Silicon | 60 | 0.0050 | 144 | 32 | 0.4 |
| | Example | | | oxide | | | | | |
| 28 | Reference | 1 | 12 | Silicon | 60 | 0.0050 | 144 | 32 | 0.4 |
| | Exemple | | | oxide | | | | | |
| 29 | Reference | 1 | 12 | Silicon | 60 | 0.0050 | 144 | 32 | 0.4 |
| | Example | | | oxide | | | | | |
| 30 | Reference | 1 | 12 | Silicon | 60 | 0.0050 | 144 | 32 | 0.4 |
| | Example | | | oxide | | | | | |
| 31 | Reference | 1 | 12 | Silicon | 60 | 0.0050 | 144 | 32 | 0.4 |
| | Example | | | oxide | | | | | |
| 32 | Reference | 1 | 12 | Silicon | 60 | 0.0050 | 144 | 32 | 0.4 |
| | Example | | | oxide | | | | | |

| | Configuration of magnetic bead | Evaluation result of magnetic bead | | | |
|---|---|---|---|---|---|
| | Metal | Nucleic | Relative | | |
| | ion | acid | evaluation | | Magnet |
| | concentration | collection | of | | separation |
| Sample | of supernatant | amount | yield | Redispersibility | rate |
| No. | ppm | ng/μL | — | — | — |
| 26 | 0.05 | 31.2 | B | — | — |
| 27 | 0.5 | 36.4 | A | — | — |
| 28 | 1.0 | 39.5 | A | — | — |
| 29 | 1.5 | 36.1 | A | — | — |
| 30 | 2.5 | 30.6 | B | — | — |
| 31 | 5.0 | 31.3 | B | — | — |
| 32 | 7.5 | 21.8 | D | — | — |

As shown in Tables 2 to 5, the magnetic beads of each Example had a favorable nucleic acid yield. As shown in Table 6, it was found that the metal ion concentration in the magnetic bead suspension significantly affects the nucleic acid collection amount. Therefore, according to the magnetic beads in each Example that can optimize an elution amount of metal ions, it was found that biological material extraction efficiency is improved.

As shown in Tables 2 to 5, the magnetic beads of each Example also had favorable redispersibility. The magnetic bead having favorable redispersibility is less likely to aggregate and thus can contribute to an increase in an amount of the adsorbed nucleic acid. Therefore, from the viewpoint of redispersibility, it can be said that the magnetic beads of each Example have high biological material extraction efficiency.

Further, the magnetic beads of each Example have a favorable magnetic separation rate. Therefore, it can be said that magnetic separation can be rapidly performed by using the magnetic beads of each Example.

What is claimed is:

1. A magnetic bead comprising:
a magnetic metal particle; and
a coating layer that covers a surface of the magnetic metal particle, contains an inorganic oxide, and has an average thickness of 15 nm or more, wherein
when the magnetic bead is subjected to
an operation of mixing 0.1 g of ethylenediaminetetraacetic acid (EDTA), 5 mL of pure water, and 100 mg of the magnetic bead to prepare a first mixture,
an operation of mixing the first mixture with 5 mL of hydrochloric acid having a concentration of 10 mM to obtain a second mixture, and then stirring the second mixture with a vortex mixer for 30 minutes, and
an operation of allowing the stirred second mixture to stand for one day, then collecting a supernatant, and measuring a metal ion concentration of the supernatant by high-frequency inductively coupled plasma (ICP) emission spectroscopy,
the metal ion concentration of the supernatant is 0.01 ppm or more and 5.00 ppm or less.

2. The magnetic bead according to claim 1, wherein
the inorganic oxide is a silicon oxide.

3. The magnetic bead according to claim 1, wherein
the magnetic metal particle is made of a Fe-based alloy.

4. The magnetic bead according to claim 3, wherein
the Fe-based alloy contains Fe as a main component and one or two or more selected from the group consisting of Cr, Si, B, C, P, Nb, and Cu.

5. The magnetic bead according to claim 1, wherein
the magnetic metal particle has an amorphous structure.

6. The magnetic bead according to claim 1, wherein
a particle diameter D50 where a cumulative value from a small diameter side in a cumulative distribution curve obtained from a volume-based particle size distribution is 50% is 0.5 μm or more and 15 μm or less.

7. The magnetic bead according to claim 6, wherein
a ratio t/D50 of an average thickness t of the coating layer to the particle diameter D50 is 0.0001 or more and 0.05 or less.

8. A magnetic bead reagent comprising:
the magnetic bead according to claim 1; and
a dispersion medium in which the magnetic bead is dispersed.
